(19) United States
(12) Patent Application Publication
(10) Pub. No.: US 2020/0337863 A1
(43) Pub. Date: Oct. 29, 2020

(54) INTERBODY DISTRACTION AND STABILISATION DEVICE

(71) Applicants: BPATH, PARIS (FR); MOOVSPINE, BORDEAUX (FR)

(72) Inventors: Richard Laurent MINFELDE, PARIS (FR); Paolo MANGIONE, BORDEAUX (FR); Istvàn HOVORKA, NICE (FR)

(21) Appl. No.: 16/955,228

(22) PCT Filed: Dec. 18, 2018
(86) PCT No.: PCT/EP2018/085660

§ 371 Date: Jun. 18, 2020

(30) Foreign Application Priority Data
Dec. 18, 2017 (FR) 17 62395

(51) Int. Cl. A61F 2/46; A61F 2/44; A61F 2/30; A61B 17/02

(52) U.S. Cl. CPC A61F 2/4611; A61F 2/447; A61F 2/30724; A61B 17/025; A61F 2/30771; A61F 2002/30841; A61F 2002/30261; A61F 2002/30556; A61F 2002/30583; A61F 2002/30593; A61F 2002/4631; A61B 2017/0256

(57) ABSTRACT

An interbody distraction and stabilisation device, comprising an implant comprising a casing for containing a filling material and a distraction element that is associated with the casing and that has opposing first and second sides, the distraction element having a first state in which the distraction element has a first height and a second state in which the distraction element has a second height greater than the first height so as to keep body parts spaced apart from each other.

The present invention relates to the field of surgery of the vertebral column.

### STATE OF THE ART

For certain pathologies of the vertebral column, such as deformations of the vertebral column and radicular pains, it is known to release neurological elements by moving vertebrae apart from each other (also referred to as “distracting”) and by implanting implants in the patient's body for the purpose of holding the vertebrae in position and stabilizing the vertebral column. The implants are for performing arthrodesis by preventing vertebrae from moving relative to each other in order to encourage bone fusion between said vertebrae.

Nevertheless, the surgery required for preventing such movement needs disinsertion of certain muscles and screws to be implanted in bony parts. The surgery is thus relatively burdensome, requires general anesthetic, and needs to be followed by a period of rehabilitation. There is also a risk of complications, in particular for people having bone structure that is weakened, specifically by osteoporosis. As a result, this kind of stabilization is not well adapted to older people.

### OBJECT OF THE INVENTION

An object of the invention is to provide means for performing such distraction in a manner that is simple, durable, and as non-traumatic as possible.

### BRIEF SUMMARY OF THE INVENTION

To this end, according invention, there is provided a device for interbody distraction and stabilization comprising an implant including a flexible envelope for receiving a filler material, and a distraction element that is associated with the envelope, having mutually-opposite first and second sides, the distraction element having a first state in which the distraction element has a first height, and a second state in which the distraction element has a second height greater than the first height, the second height being measured along a direction normal to a third side extending between the first and second sides, the third side and a fourth side that is opposite to the third side being arranged to bear against body parts of the patient in such a manner as to keep them spaced apart from each other substantially by the second height when the distraction element is in its second state.

The body parts are thus held spaced apart from each other by combined action of the distraction element and of the filler material contained in the envelope. The filler material may be a cement, aggregate, or a particulate material that is suitable for agglomerating in order to withstand compression forces. The device of the invention makes it possible to form a rigid block in the flexible envelope that is suitable for holding the body parts a predetermined distance apart.

While in its first state, the distraction element together with the envelope for confining the filler material is easier to insert between the body parts, and distraction is obtained when the distraction element is taken from its first state to its second state.

Advantageously, the distraction element is in the form of a cage and the envelope is designed to extend in a central space of the cage, the first and second sides being open to enable two lateral portions of the envelope to extend outside the cage once the filler material is inserted in the envelope.

The envelope is folded inside the cage and its flexibility enables it to be unfolded progressively as the filler material is inserted therein. In a variant, the flexible envelope, made of a stretchable material, may be in a rest state inside the cage and may expand towards a stretched state progressively as the filler material is inserted therein.

When applied to a vertebral column, the distraction element is inserted between two vertebrae in order to space them apart from each other so as to increase the height and the volume of the disk space, e.g. in order to provide a space between the vertebrae having the thickness of about one disk, the confinement envelope is then deployed in said space, and filler material is inserted into the confinement envelope. The filler material may be a cement so that once solidified the block of cement maintains the intervertebral space.

Then advantageously, the confinement envelope is a closed envelope.

The confinement envelope then serves either to keep in place a material that is to acquire cohesion, e.g. by solidifying, or else itself to maintain the cohesion of said material (if it is a divided solid). In a variant, the envelope may be open when the filler material is designed to solidify quickly.

In another version of the invention, the device includes a sealing element that is complementary to the confinement envelope and that is arranged to provide sealing at a cut made in the vertebral fibrous ring in order to enable the implant to be inserted between two vertebrae.

This sealing element is removed after the envelope has been filled, while the cage and the envelope remain in place. The size of the sealing element may be defined so as to protect sealing around the point of insertion into the fibrous ring, or indeed further in order to protect the posterior disk wall or even the foramen wall.

It is then preferable for the sealing element to be in the form of a skirt surrounding a portion of a tube for inserting the filler material between the vertebrae and initially connected to the confinement envelope.

In a first embodiment of the implant, the first height is measured along a direction normal to the first side.

Thus, the distraction element is caused to go from its first state to its second state by being pivoted about an axis perpendicular to the normal direction.

In a second embodiment of the implant, the first height is measured along a direction normal to the third side, and the distraction element is arranged to be plastically deformable between its first and second states.

Distraction is then the result of the distraction element being deformed to take up its second state.

In a first variant of the second embodiment, the third and fourth sides are connected together by a cuff that holds a first edge of the third side away from a first edge of the fourth side, the third and fourth sides each having a respective second edge opposite from its first edge, the second edges being close to each other when the distraction element is in its first state and spaced apart from each other when the distraction element is in its second state.

In a second variant of the second embodiment, the distraction element contains relatively rigid internal reinforcement having a first end fastened to a fifth side of the distraction element and an opposite second end spaced apart from a sixth side of the distraction element when the distraction element is in its first state, the sixth side of the distraction element being movable towards the second end of the reinforcement by causing the third and fourth sides of the distraction element to bulge outwards in order to bring the distraction element into its second state.

This reinforcement contains all or part of the filler envelope for the filler material and its shape enables it to be deployed in the disk space.

Preferably, the sixth side of the distraction element is caused to move towards the second end of the reinforcement by means of a tool having a bearing portion bearing against the sixth side and a tie that is movable relative to the bearing portion and that has both an end for attachment to the second end of the reinforcement and also a handling end.

Preferably, each of the third and fourth sides is provided with at least one crampon spike arranged to penetrate into the body parts against which the third and fourth sides bear respectively.

This improves retention of the implant in place.

According to an advantageous characteristic of the invention, the confinement envelope includes external reinforcement preventing it from being stretched beyond a certain size.

According to another advantageous characteristic, the confinement envelope dissolves under the effect of heat (e.g. while the cement is polymerizing).

In a variant implementation, the pipe or tube used for injecting the filler material into the confinement envelope is initially integral with the confinement envelope and is subsequently detached therefrom.

Other characteristics and advantages of the invention appear on reading the following description of particular, nonlimiting embodiments of the invention.

### BRIEF DESCRIPTION OF THE FIGURES

Reference is made to the accompanying drawings, in which:

FIG. 1 is a perspective view of an implant in a first embodiment of the invention, in its first state and in position between two vertebrae;

FIG. 2 is a view analogous to FIG. 1 showing the implant in its second state;

FIG. 3 is a perspective view of said implant from another angle;

FIG. 4 is a view analogous to FIG. 1 showing the implant after the confinement envelope has been deployed and cement has been inserted;

FIG. 5 is a view analogous to FIG. 1 showing the implant with the fibrous ring being visible;

FIG. 6 is a perspective view of an implant in a second embodiment of the invention, in its first state;

FIG. 7 is a view analogous to FIG. 6 showing the implant in its second state;

FIG. 8 is a perspective view of said implant in its second state and in position between two vertebrae;

FIG. 9 is a perspective view showing the tool for causing the implant to go from its first state to its second state;

FIG. 10 is a perspective view of an implant in a third embodiment of the invention, in its first state;

FIG. 11 is a view analogous to FIG. 10 showing the implant in its second state, with its filling envelope cut away and shown empty;

FIG. 12 is a perspective view of an implant in a variant of the third embodiment of the invention, in its first state;

FIG. 13 is a view analogous to FIG. 12 showing the implant in its second state;

FIG. 14 is a perspective view of an implant in a fourth embodiment of the invention, in its first state;

FIG. 15 is a view analogous to FIG. 12 showing the implant in its second state;

FIG. 16 is a perspective view showing the use of a sealing skirt while inserting the filling material.

### DETAILED DESCRIPTION OF THE INVENTION

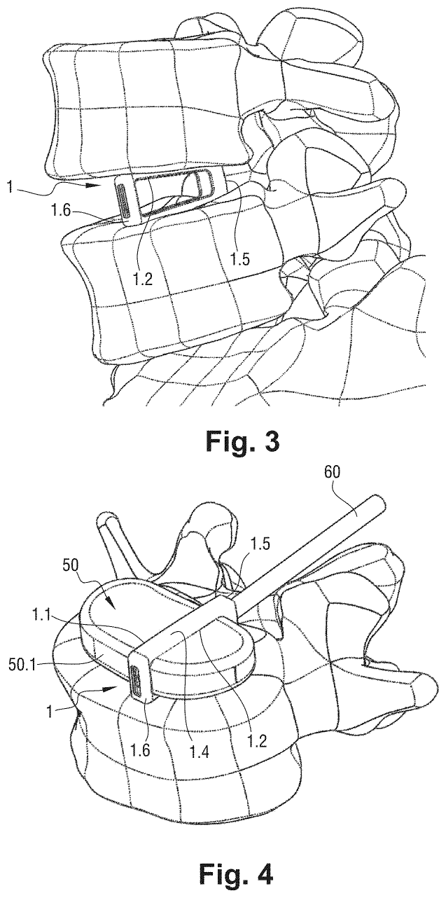

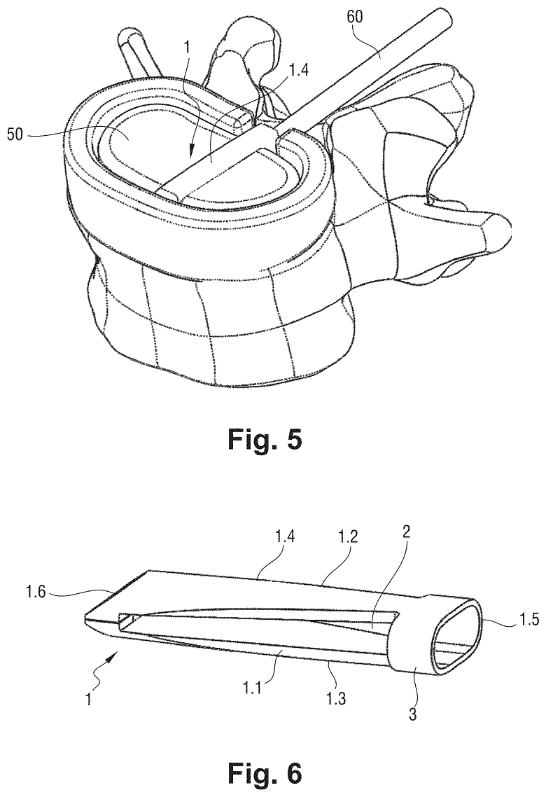

The figures show only those portions of the vertebral column that are useful for describing invention or portions that do not impede visibility of the invention. The upper vertebra is shown in FIG. 3 only. The fibrous ring (or annulus fibrosus) of the intervertebral disk is thus shown in FIG. 5, even though it is not removed for putting the device into place, with its posterior portion merely being cut open to pass instruments or tools, and also to put the implant into place, as explained below.

With reference to the figures, the device of the invention for interbody distraction and stabilization comprises a distraction member in the form of an implant having a distraction element in the form of a cage, given overall reference 1, that is arranged to be inserted between two vertebrae of a patient in order to hold them apart from each other, and a cement confinement envelope 50 that is arranged to be inserted empty between the two vertebrae. The envelope 50 is not shown in some of the figures, mainly in order to provide better visibility of other portions of the implant.

The confinement envelope 50 is a closed envelope made of flexible material having thickness lying in the range 0.5 millimeters (mm) to 1.5 mm approximately, and it is elastically deformable in this example. By way of example, the confinement envelope 50 may be made by braiding threads of polyethylene, of polypropylene, of polyester, of polyurethane, of polyamide, or of any braidable material that is well tolerated biologically. The confinement envelope 50 could equally well be a single piece of silicone, or of a derivative of polylactic acid, or indeed of various polymers that are well tolerated biologically. The confinement envelope 50 may combine portions that are woven with portions that are not woven, by using a technique for combining materials by injection, by molding, by gumming, or in some other way.

The cage 1 defines a space 2 for receiving at least a central portion of the confinement envelope 50. The cage 1 comprises:
mutually opposite first and second sides 1.1 and 1.2 that are open to allow two lateral portions of the confinement envelope 50 to extend out from the cage 1;
mutually opposite third and fourth sides 1.3 and 1.4 that are solid and each of which is to bear against a respective one of the two vertebrae, the third and fourth sides 1.3 and 1.4 extending between the first and second sides 1.1 and 1.2;
fifth and sixth sides 1.5 and 1.6 that extend respectively between a first edge of the third side 1.3 and a first edge of the fourth side 1.4 and between a second edge of the third side 1.3 and a second edge of the fourth side 1.4.

The cage 1 has a first state in which the cage 1 has a first height, and a second state in which the cage 1 has a second height greater than the first height, the second height being measured along a vertical direction while the cage 1 is resting on its third side 1.3. Each of the third and fourth sides 1.3 and 1.4 is for bearing against a respective one of the two vertebrae so as to hold them spaced apart from each other at the second height when the cage 1 is in its second state. The third and fourth sides 1.3 and 1.4 are shown as being solid, however they could include one or more openings, so long as the openings do not prevent the third and fourth sides from bearing against the vertebrae.

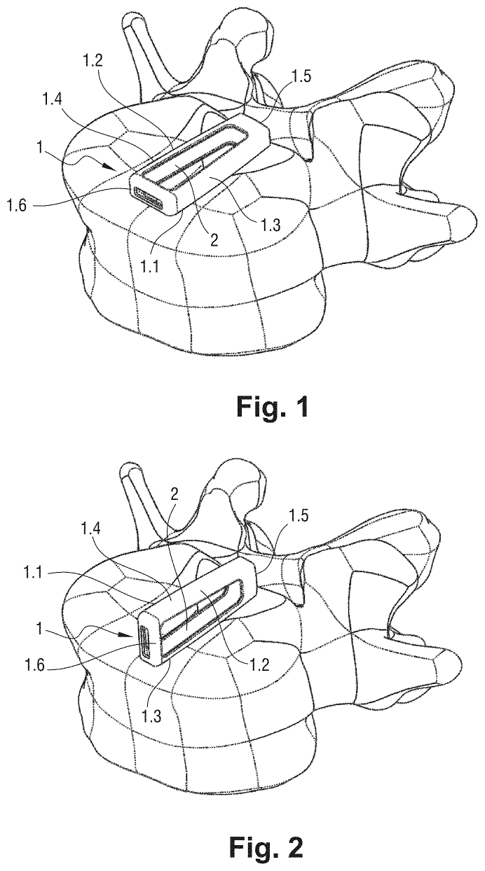

With reference to FIGS. 1 to 5, the first height is measured along a direction that is normal to the first side 1.1 or the second side 1.2.

In this embodiment, the cage 1 is substantially in the shape of a rectangular parallelepiped with the sides 1.3 and 1.4 converging a little towards the side 1.5, it is rigid, and it is inserted between the two vertebrae that are to be spaced apart with its first and second sides facing towards said vertebrae, i.e. while it is in its first state (see FIG. 1). For this purpose, a tool (not shown) is used that has a handle with a free end screwed into the fifth side 1.5. Thereafter, the cage 1 is pivoted (in the screw-tightening direction of the tool) to bring the third and fourth sides 1.3 and 1.4 face-to-face with respective ones of the vertebrae, thereby spacing said vertebrae apart from each other (see FIGS. 2 and 3). The tool is then unscrewed.

In this embodiment, the confinement envelope is inserted and folded inside the cage 1 before the cage is inserted between the vertebrae. In a first configuration, the envelope 50 received in the cage 1 is a closed envelope with an access for enabling the filler material to be inserted therein, said access either being left open or else being closed after the filler material has been inserted. In a second configuration, the envelope is provided with a removable skirt.

In the first configuration, and as shown in figures, the envelope 50 has a filler orifice in which one end of a cement feed pipe 60 is engaged. The envelope 50 is inserted in the cage 1 so as to be subsequently filled with cement or with a biomaterial, thereby causing it to deploy out from the cage 1 by passing through the first and second sides 1.1 and 1.2 so as to fill a large portion of the intervertebral space. Only the central portion 50.1 of the envelope 50 then remains in the cage 1. FIG. 4 shows the envelope 50 fully deployed, and FIG. 5 shows the envelope 50 surrounded by the fibrous ring with only its posterior portion being cut so as to enable the implant and the envelope to be put into place between the vertebrae. The deployed envelope 50 then covers the majority of the facing surfaces of the end-plates of the adjacent upper and lower vertebrae, with the cage 1 itself covering only a minority surface area (preferably less than one-third of the surface area of the deployed envelope 50).

In the second situation, the envelope 50 possesses a skirt-shaped element at the filler orifice, which element is placed at the periphery of the intervertebral space in order to come into the vicinity of or in contact with the edges of the vertebrae and of the fibrous ring in order to prevent the cement from leaving the envelope 50. Once the envelope 50 is in place, there remains a space for inserting the end of the cement feed pipe 60.

Cement is then inserted into the envelope 50 via the cement feed pipe 60.

In the description below of second, third, and fourth embodiments, elements that are identical or analogous to those described above are given numerical references that are identical.

In these embodiments, the first and second heights are both measured in a direction normal to the third side 1.3 and the cage 1 is arranged to be plastically deformable between its first and second states.

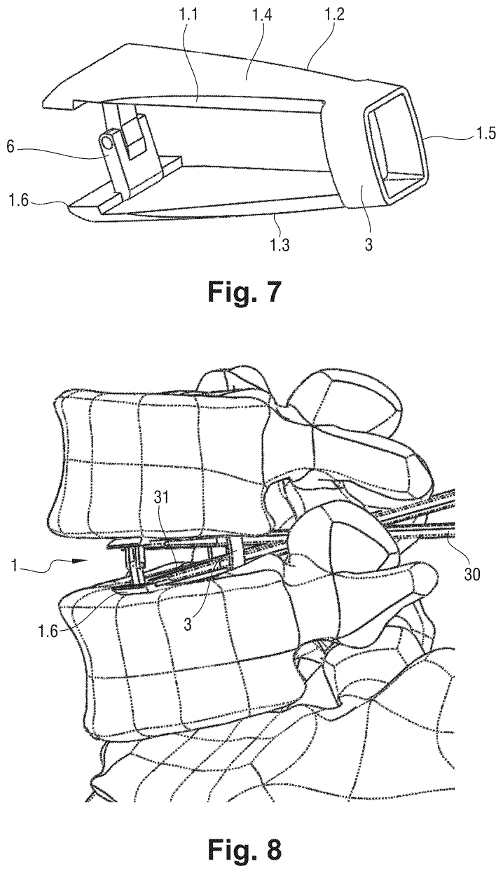

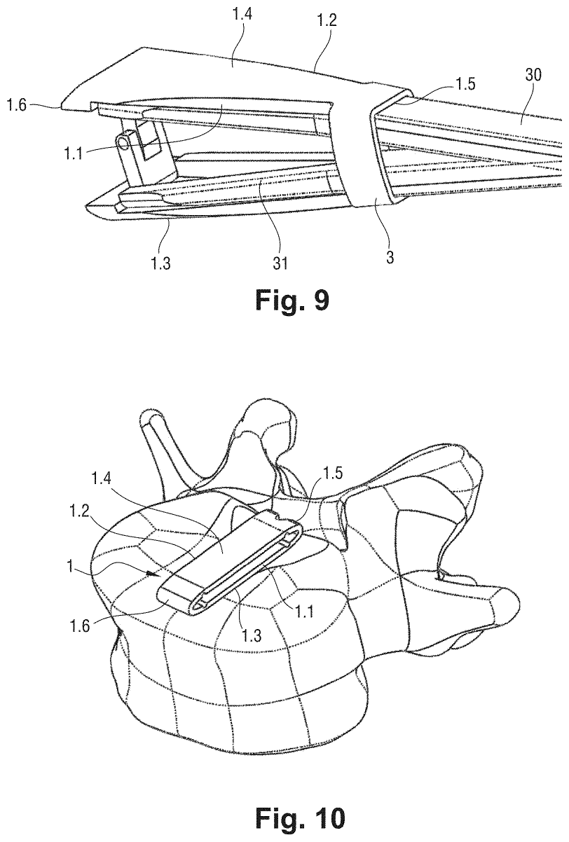

In the second embodiment, as shown in FIGS. 6 to 9, the third and fourth sides 1.3 and 1.4 are connected together by a cuff 3 that holds the first edge of the third side 1.3 spaced apart from the first edge of the fourth side 1.4. The second edges of the third and fourth sides are close to each other when the cage 1 is in its first state, and spaced apart from each other when the cage 1 is in its second state. In this example, the cage 1 is wedge-shaped while it is in its first state, with the sixth side being greatly flattened.

In this embodiment, the cage 1 is inserted between the two vertebrae that are to be spaced apart (or “distracted”), with its third and fourth sides 1.3 and 1.4 facing towards respective ones of said vertebrae, i.e. in its first state (see FIG. 6). The sixth side 1.6 is inserted first so that the wedge shape of the cage 1 in its first state makes it easier to insert the cage 1 between the two vertebrae.

The cage 1 is inserted into the intervertebral space by means of a tool 30 having one end 31 that is engaged inside the cage 1 by passing through the cuff 3.

Said end 31 of the tool 30 comprises two fork portions that can be spaced apart from each other in order to move the second edges of the third and fourth sides 1.3 and 1.4 away from each other. The branches of each fork portion can be moved towards each other for insertion into the cage 1 and for removal from the cage 1, or they can be moved apart from each other in order to distribute forces while deforming the cage 1. Subsequently, the tool 30 is removed after being closed.

Preferably, a support element 6 is provided that is arranged between the mutually spaced-apart second edges in order to oppose them moving towards each other. In this example, the support element 6 is a deployable element, such as a toggle, which makes it easier to insert in the cage 1. The support element 6 could also be a single piece.

Once the cage 1 has been deformed and the support element 6 is in place, the envelope 50 is inserted into the central space, e.g. through the cuff 3, and the filler material is inserted into the envelope 50 as in the first embodiment.

It should be observed that the cuff 3 is also deformed in a lengthwise direction when the cage 1 is in its second state.

In a variant, the tool 30 may be much simpler. The end 31 then has only two generally-rectangular portions, each of which comprises a single piece. It is also possible to envisage making use of rectangular blocks that are forcibly slid into the cage 1, one on another.

In the variant of FIG. 16, use is made of a cage 1 in compliance with the second embodiment (the confinement envelope 50 is not shown) together with a sealing element arranged to provide sealing at the cut made in the vertebral fibrous ring in order to enable the cage 1 to be inserted between the two vertebrae.

The sealing element is in the form of a skirt 51 surrounding a portion of a tube 52 for inserting the filler material between the vertebrae. Sealing is provided between the fibrous ring, the vertebrae, and the tube 52.

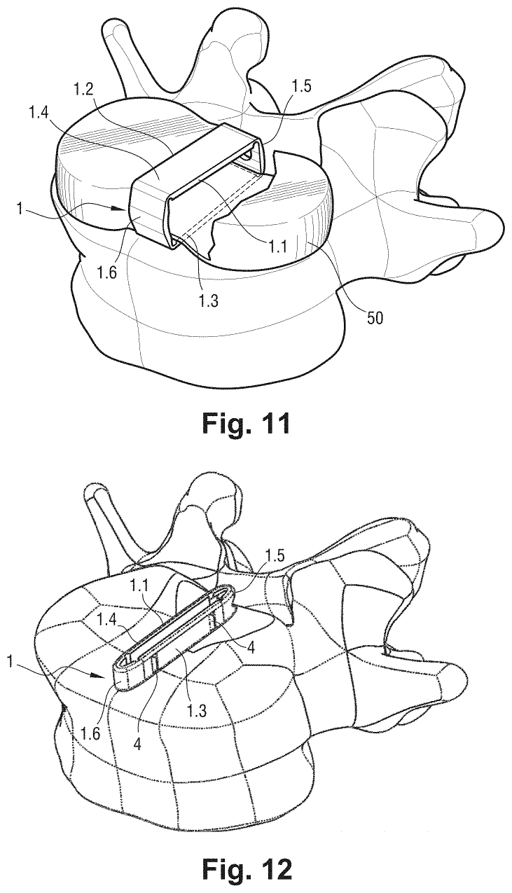

In the third embodiment, as shown in FIGS. 10 and 11, each of the fifth and sixth sides 1.5 and 1.6 has a respective central portion that is folded in such a manner that the fifth and sixth sides 1.5 and 1.6 are folded in half outwards from the cage 1 when the cage 1 is in its first state (FIG. 10).

In this embodiment, the cage 1 is inserted between the two vertebrae that are to be distracted with its third and fourth sides 1.3 and 1.4 facing towards respective ones of said vertebrae (FIG. 10). The fifth and sixth sides 1.5 and 1.6 of the cage 1 are unfolded by means of a tool (not shown) comprising two telescopic portions, namely a portion for bearing against the central portion of the fifth side 1.5 and a tie passing through a hole made in the central portion of the fifth side 1.5 and having an end attached to the central portion of the sixth side 1.6. By pulling on a handle end of the tie, opposing forces are exerted on the fifth and sixth sides 1.5 and 1.6, and it can be understood that the tie straightens the sixth side 1.6, while the bearing portion straightens the fifth side 1.5. The cage 1 is then in its second state (FIG. 11).

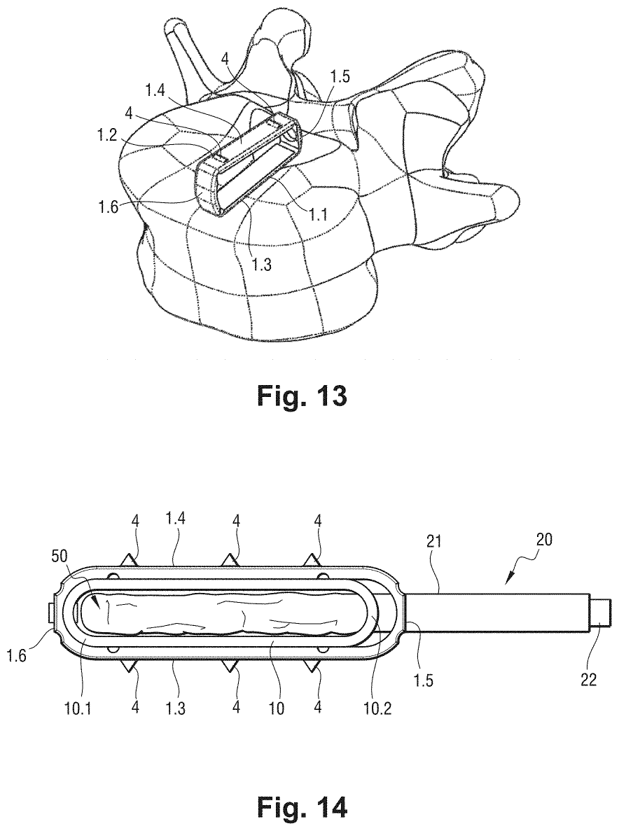

In the variant of FIGS. 12 and 13, the cage 1 is identical to the cage of FIGS. 10 and 11, but it is inserted between the two vertebrae that are to be spaced apart with its first and second sides facing towards respective ones of said vertebrae (see FIG. 12). For this purpose, use is made of a first tool (not shown, but identical to the tool used in the first embodiment) comprising a handle with a free end screwed into the fifth side 1.5.

The cage 1 is then pivoted (in the screw-tightening direction of the tool) to cause the third and fourth sides 1.3 and 1.4 to face respective ones of the two vertebrae. The first tool is then unscrewed and a second tool (identical to the tool of the third embodiment) is used to unfold the sixth side and the fifth side 1.5. The cage 1 is then in its second state (FIG. 13).

The envelope 50 is put into place after removing the second tool. As above, it is possible to put the confinement envelope in the cage 1 before inserting it between the vertebrae.

It should be observed that each of the third and fourth sides 1.3 and 1.4 is provided with crampon spikes 4 projecting from said sides towards the outside of the cage 1 in order to penetrate into the vertebrae against which the third and fourth sides 1.3 and 1.4 bear respectively.

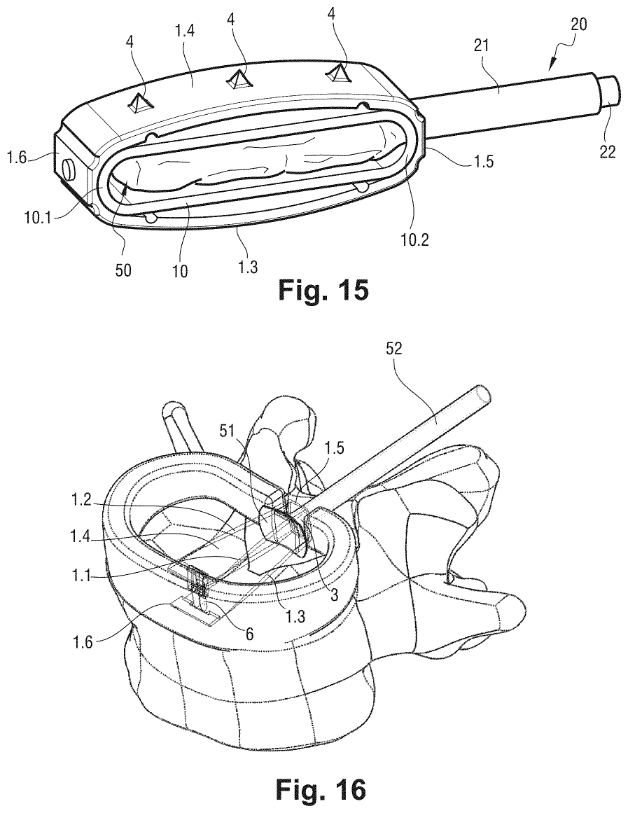

In the fourth embodiment, as shown in FIGS. 14 and 15, the cage 1 contains relatively rigid internal reinforcement 10 that is likewise in the shape of a cage, but of smaller dimensions than the cage 1. The internal reinforcement 10 contains the envelope 50 for filling and it has a first end 10.1 fastened to the sixth side 1.6 of the cage 1 and an opposite second end 10.2 spaced apart from the fifth side 1.5 of the cage 1 when the cage 1 is in its first state (FIG. 14). Moving together the fifth side 1.5 of the cage 1 and the second end 10.2 of the reinforcement 10 causes the third and fourth sides 1.3 and 1.4 of the cage 1 to bulge outwards, and brings the cage 1 into its second state (FIG. 15) by plastic deformation. In order to avoid the third and fourth sides 1.3 and 1.4 of the cage 1 sagging towards the inside of the cage 1, the internal reinforcement 10 presents a dimension, measured in a direction normal to the third and fourth sides, that is substantially equal to the distance between the third and fourth sides, such that the internal reinforcement 10 opposes sagging of the third and fourth sides 1.3 and 1.4 of the cage 1 towards the inside of the cage 1.

The fifth side 1.5 of the cage 1 and the second end 10.2 of the reinforcement 10 are caused to move together by means of a tool, given overall reference 20, having a bearing portion 21 for bearing against the fifth side 1.5 and a tie 22 that is movable relative to the bearing portion 21 and that has one end for attaching to the second end 10.2 reinforcement 10 and a handling end that is not shown. In this example, the bearing portion 21 is made up of a tube in which the tie 22 slides telescopically.

It should be observed that each of the third and fourth sides 1.3 and 1.4 is provided with crampon spikes 4 projecting from said sides towards the outside of the cage 1 in order to penetrate into the vertebrae against which the third and fourth sides 1.3 and 1.4 bear respectively.

Naturally, the invention is not limited to the embodiments described and covers any variant coming within the ambit of the invention as defined by the claims.

The material of the confinement envelope 50 need not be stretchable. The confinement envelope 50 may include non-stretchable outer reinforcement, e.g. a net, in order to limit expansion of the envelope 50 beyond a certain dimension. The net may extend throughout the envelope in order to prevent expansion in all directions, or it may extend in a portion only of the envelope in order to limit expansion locally in one or more directions.

It is possible for the cage to be elastically deformable between its two states. It is then necessary to provide means for maintaining its deformed state. For example, in the fourth embodiment, a screw may be provided that passes through the fifth side of the cage 1 and that is screwed into the second end of the reinforcement 10 in order to cause the fifth side to move towards the second end of the reinforcement 10.

Naturally, other advantageous characteristics or variants can be devised:
the device may include a sealing element arranged to provide sealing at a cut made in the vertebral fibrous ring in order to enable the implants to be inserted between two vertebrae;
the confinement envelope 50 may be made of a material that degrades under the effect of heat (e.g. while the cement is polymerizing) so that the envelope 50 disappears after a certain length of time;
the envelope 50 may be made of a resorbable material, e.g. from polylactic acid and/or from polyglycolic acid;
the pipe used for injecting the filler material into the envelope may have one end connected to the confinement envelope before the implant is put into place, and may subsequently be detached.

The envelope may be made up of two portions, e.g. in the form of pouches, that are fastened to the edges of the cage. Said portions may be sewn, welded, or adhesively bonded in continuous manner with the cage 1 so that together they define a volume.

The envelope need not necessarily be hermetically sealed in that it may leave an access for filling it with the filler material.

The envelope may extend from only one side of the cage.

Whatever the embodiment, the envelope may be put into place in the distraction element before or after the distraction element is put into place between the vertebrae.

The cage 1 may also be arranged in the envelope 50 before the resulting assembly is put into place between the vertebrae. The cage then bears against the vertebrae indirectly via the portions of the envelope that extend between said vertebrae and the third and fourth sides, respectively. The distraction element need not be in the form of a cage and need not have sides that are open.

1. A device for interbody distraction and stabilization comprising an implant including a flexible confinement envelope for containing a filler material, and a distraction cage that defines a central space for receiving the envelope and that includes mutually-opposite first and second sides, the distraction cage having a first state in which the distraction cage has a first height, and a second state in which the distraction cage has a second height greater than the first height in order to give rise to distraction of body parts when passing from its first state to its second state, the second height being measured along a direction normal to a third side extending between the first and second sides, the third side and a fourth side that is opposite to the third side being arranged to bear against the body parts of the patient in such a manner as to keep them spaced apart from each other substantially by the second height when the distraction cage is in its second state, the first and second sides being open to enable two lateral portions of the envelope to extend outside the cage once the filler material is inserted in the envelope after the distraction cage has been put into its second state so as to occupy the space released by the distraction cage spacing apart the body parts and so as to keep the body parts spaced apart from each other by combined action of the distraction cage and of the filler material contained in the envelope.

2. (canceled)

3. (canceled)

4. The device according to claim 1, including a sealing element arranged to provide sealing at a cut made in the vertebral fibrous ring in order to enable the implant to be inserted between two vertebrae.

5. The device according to claim 4, wherein the sealing element is in the form of a skirt surrounding a portion of a tube for inserting the filler material between the vertebrae.

6. The device according to claim 1, wherein the first height is measured along a direction normal to the first or second side.

7. The device according to claim 1, wherein the first and second heights are measured along a direction normal to the third side, and the distraction cage is arranged to be deformable between its first and second states.

8. The device according to claim 7, wherein the distraction cage is arranged to be plastically deformable between its first and second states.

9. The device according to claim 8, wherein the third and fourth sides are connected together by a cuff that holds a first edge of the third side away from the first edge of the fourth side, the third and fourth sides each having a respective second edge opposite from its first edge, the second edges being close to each other when the distraction cage is in its first state and spaced apart from each other when the distraction cage is in its second state.

10. The device according to claim 9, including a support element arranged between the spaced-apart second edges in order to oppose them moving towards each other.

11. The device according to claim 7, wherein the distraction cage contains relatively rigid internal reinforcement having a first end fastened to a fifth side of the distraction cage and an opposite second end spaced apart from a sixth side of the distraction cage when the distraction cage is in its first state, the sixth side of the distraction cage being movable towards the second end of the reinforcement by causing the third and fourth sides of the distraction cage to buckle outwards in order to bring the distraction cage into its second state.

12. The device according to claim 11, wherein the sixth side of the distraction cage is caused to move towards the second end of the reinforcement by means of a tool having a bearing portion bearing against the sixth side and a tie that is movable relative to the bearing portion and that has both an end for attachment to the second end of the reinforcement and also a handling end.

13. A The device according to claim 1, wherein each of the third and fourth sides is provided with at least one crampon spike arranged to penetrate into the body parts against which the third and fourth sides bear respectively.
